(19) United States
(12) Patent Application Publication
(10) Pub. No.: US 2008/0154312 A1
(43) Pub. Date: Jun. 26, 2008

(54) ACTIVE SETTLING PLATE WITH ELASTOMERIC MEMBERS AND METHOD OF USE

(76) Inventors: Dennis Colleran, North Attleboro, MA (US); James Spitler, Plano, TX (US)

Correspondence Address: CARR LLP (IST), 670 FOUNDERS SQUARE, 900 JACKSON STREET, DALLAS, TX, 75202, US

(21) Appl. No.: 11/955,077

(22) Filed: Dec. 12, 2007

Related U.S. Application Data
(60) Provisional application No. 60/869,577, filed on Dec. 12, 2006.

(51) Int. Cl. A61B 17/58

(52) U.S. Cl. 606/283; 606/280

(57) ABSTRACT

Disclosed are certain embodiments of a bone a surgical bone plate system which may include a first plate member having a first and second laterally offset coupling members and a receiving segment with a longitudinal portal. The surgical bone plate system may incorporate a second plate member having a third and fourth laterally offset coupling members, and a slider segment dimensioned to fit within the longitudinal portal of the first plate member. A first elastomeric member may couple to the first and third laterally offset coupling members and a second elastomeric member may extend between and couple to the second and fourth laterally offset coupling members. The first and second elastomeric members may be laterally spaced apart from the receiving segment and the slider segment to define a first and second windows.

### CROSS REFERENCE TO RELATED APPLICATIONS

This application relates to, and claims the benefit of the filing date of: co-pending U.S. provisional patent application Ser. No. 60/869,577, filed Dec. 12, 2006, entitled ACTIVE SETTLING PLATE AND METHOD OF USE, which is hereby incorporated by reference.

### TECHNICAL FIELD

The invention relates generally to instruments and methods for spinal surgery and, more particularly, to cervical plating systems and instruments for stabilizing and/or fusing the spine.

### BACKGROUND INFORMATION

The human spine is a complex structure designed to achieve a myriad of tasks, many of them of a complex kinematic nature. The spinal vertebrae allow the spine to flex in three axes of movement relative to the portion of the spine in motion. These axes include the horizontal (bending either forward/anterior or aft/posterior), roll (lateral bending to either left or right side) and rotation (twisting of the shoulders relative to the pelvis).

The spine of most human adults consists of 24 connected bones called vertebrae. The cervical vertebrae begin at the base of the skull. Seven vertebrae make up the cervical spine, which are abbreviated C1, C2, C3, C4, C5, C6 and C7. The cervical vertebrae are smaller in size compared to other spinal vertebrae. The purpose of the cervical spine is to contain and protect the spinal cord, support the skull, and enable a wide range of head movement. The vertebrae allow the head to rotate side to side, bend forward and backward.

The intervertebral spacing (between neighboring vertebrae) in a healthy spine is maintained by a compressible and somewhat elastic disc. The disc serves to allow the spine to move about the various axes of rotation and through the various arcs and movements required for normal mobility. The elasticity of the disc maintains spacing between the vertebrae, allowing room or clearance for compression of neighboring vertebrae, during flexion and lateral bending of the spine. In addition, the disc allows relative rotation about the vertical axis of neighboring vertebrae, allowing twisting of the shoulders relative to the hips and pelvis. Clearance between neighboring vertebrae maintained by a healthy disc is also important to allow nerves from the spinal cord to extend out of the spine, between neighboring vertebrae, without being squeezed or impinged by the vertebrae.

Frequently cervical spine disorders require surgery to relieve painful symptoms. One of the contributing factors associated with most spine disorders is the dehydration of the intervertebral disks, which act as a cushion between adjacent vertebrae. Over time these disks can dry out and become flattened, causing the vertebrae to lose height and its healthy resilience. The degeneration of the disks allow the vertebrae get closer together and cause nerve irritation, which usually stems from a ruptured disc, bone spurs or stenosis. Vertebral motion (neck movement) results in chronic pain.

Cervical fusion has become an accepted procedure to relieve the pressure on one or more nerve roots, or on the spinal cord. It involves the stabilization of two or more vertebrae by locking (fusing) them together in a desired spacing and orientation. The fusion restores the proper distance between the vertebrae thus preventing nerve irritation.

The cervical spine may be approached by the surgeon anteriorly, which refers to the front of the patient. The surgeon reaches the cervical spine through a small incision in the front of the neck. After retracting neck muscles, the surgeon often removes the affected intervertebral disk, which takes the pressure off the nerves or spinal cord. This is procedure is known as decompression. The surgeon then may replace the removed disk with a bone graft or interbody fusion device (such as a cage) to aid in the fusion of adjacent vertebrae and restores the distance between the vertebrae. The surgeon then may use various types of plates which provide extra force on the graft (or interbody fusion device) and support the neck to ensure that the bones fuse adequately. Holes may be drilled or tapped in the bone to allow for attachment of a plate using a bone screw or other fastener. Some screws are self tapping and may not require either taping or drilling. The plate is placed against two or more adjacent vertebrae and bone fasteners are used to secure the plate in place.

One of the problems associated with the fusion of cervical vertebrae is the tendency of the screws or other fasteners to loosen over time. As the fasteners or screws loosen the plate is not able to support or maintain the proper orientation of the vertebrae. The plate and other associated implants, which are no longer secure, can cause irritation and even trauma to local tissue structures. Another problem associated with the fusion of cervical vertebrae is the tendancy of the bones or vertebrae not to fuse together. Poor fusion may also result from subsidence of the bone graft or interbody device. Subsidence occurs when the bone graft or interbody device that is placed between to vertebral end plates sinks or settles into the vertebral end plates. When subsidence occurs, the extra force or pressure placed on the interbody device or bone graft by a plate may be reduced to nothing. If little load is transferred to the bone (or bone graft), the bone may become weaker, resulting in a poor fusion.

Various features and implants have been developed to prevent the backing out of different types of fasteners from their respective plates. These improvements usually require additional components or features which may also become loose. Additional components also increase the time and complexity of the procedure for the surgeon.

What is needed, therefore, is a system and method, which facilitates overcoming one or more of the aforementioned problems as well as other problems and to provide a device that has unique features that will facilitate reducing the risk associated with surgeries and advance the present state of the art.

### BRIEF DESCRIPTION OF THE DRAWINGS

FIG. 1. is an anterior view illustrating one possible embodiment of a plate of the present disclosure;

FIG. 2. is a cross sectional side view of the plate of FIG. 1;

FIG. 3A is a cross sectional side view taken along the line A-A in FIG. 1 illustrating one possible embodiment of a first position of the plate of the present disclosure;

FIG. 3B is a cross sectional side view taken along the line A-A in FIG. 1 illustrating one possible embodiment of a second position of the plate of the present disclosure;

FIG. 4A. is an anterior view illustrating another possible embodiment of a plate of the present disclosure;

FIG. 4B is a cross sectional side view taken along the line B-B in FIG. 4A;

FIG. 5 is an anterior view illustrating yet another possible embodiment of a plate of the present disclosure; and

FIG. 6 is an anterior view illustrating another possible embodiment of a plate of the present disclosure; and

FIG. 7 is a top view illustrating one possible embodiment of a surgical kit which may incorporate the plate of FIG. 5.

### DETAILED DESCRIPTION

For the purposes of promoting an understanding of the principles of the present inventions, reference will now be made to the embodiments, or examples, illustrated in the drawings and specific language will be used to describe the same. It will nevertheless be understood that no limitation of the scope of the invention is thereby intended. Any alterations and further modifications in the described embodiments, and any further applications of the principles of the inventions as described herein are contemplated as would normally occur to one skilled in the art to which the invention relates.

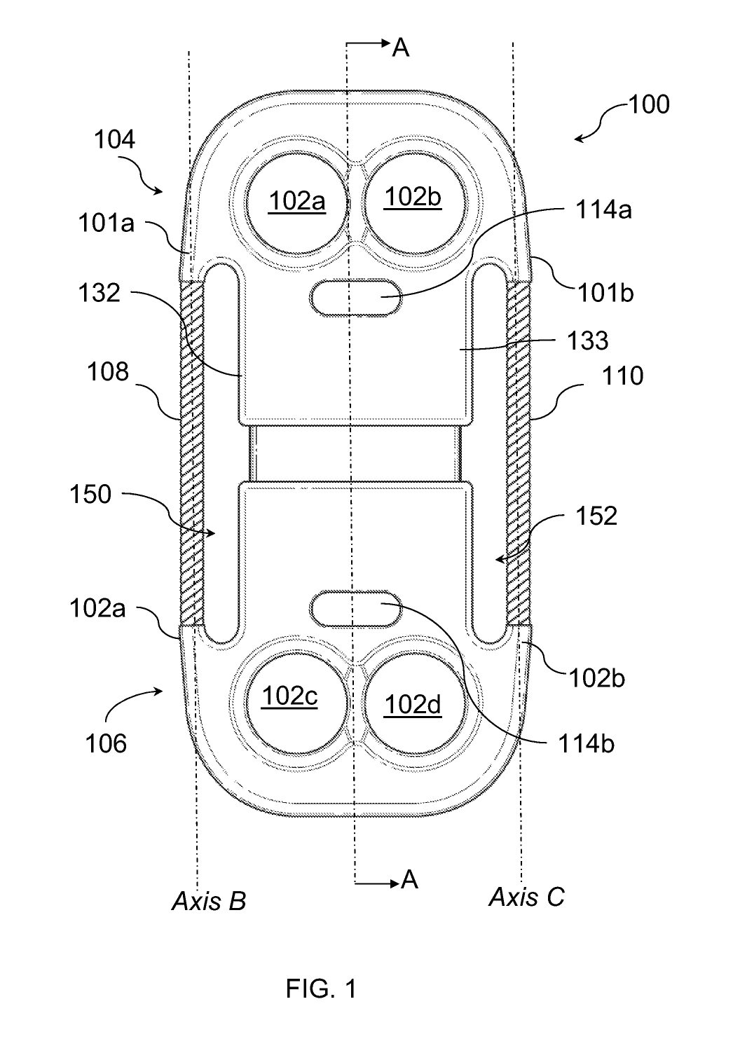

Turning now to FIG. 1, there is presented a front view of one possible embodiment of a plate 100. The plate 100 may extend generally along a longitudinal axis A-A and may include a first plate member 104, a second plate member 106 and one or more elastomeric members 108 and 110. The first and second plate members 104 and 106 may have one or more instrumentation slots 114a and 114b dimensioned to receive a plate holding instrument (not shown) which may be used expand the first and second plate members 104 and 106 apart. As will be discussed in greater detail below, the plate 100 may be fastened to one or more adjacent (or non adjacent) boney structures, such as vertebrae. The first plate and second plate member 104 and 106 may define a plurality bores 102a, 102b, 102c and 102d. The plurality of bores 102a, 102b, 102c and 102d may be dimensioned to receive a plurality of bone anchors (not shown) to secure the plate 100 to a boney structure. The elastomeric members 108 and 110 may aid in compressing a graft or an implant located between the adjacent vertebrae to promote fusion. The plate 100 may also be used to stabilize boney fractures which may or may not have an implant or graft in-between to promote fusion or improve healing. For example, the elastomeric members 108 and 110 may force long bone structures on opposing sides of a fracture together to promote fusion.

In certain embodiments a proximal end portion of the elastomeric member 108 may be secured to a first coupling member 101a that is coupled to and laterally offset from the first plate member 104. A distal end portion of the elastomeric member 108 may be secured to a second coupling member 102a that is coupled to and laterally offset from the second plate member 106. In certain embodiments a proximal end portion of the elastomeric member 110 may be secured to a third coupling member 101b that is coupled to and laterally offset from the first plate member 104. A distal end portion of the elastomeric member 110 may be secured to a fourth coupling member 102b that is coupled to and laterally offset from the second plate member 106. In certain embodiments the coupling members 101a, 101b, 102a and 102b may be a separate component or may be integral with the respective first and second plate members 104 and 106.

The first and second elastomeric members 108 and 110 may extend longitudinally along an Axis B and an Axis C that are generally parallel to the longitudinal axis A-A of the plate. The first and second elastomeric members 108 and 110 may be laterally spaced apart from the first and second plate members 104 and 106. The first elastomeric member 108 and the first and second plate members 104 and 106 may form a continuous wall that defines a first window 150. The second elastomeric member 110 and the first and second plate members 104 and 106 may form a continuous wall that defines a second window 152. The first and second windows 150 and 152 may enable a surgeon to have a better view of a graft or implant (not shown) which may be positioned between a first and second vertebrae (not shown) and determine if the plate, graft and/or implant has been placed correctly. Post operatively a surgeon may take an X-ray of the plate 100 and a boney structure, such as vertebrae, to determine the progress of fusion or healing. The first and second windows 150 and 152 may enable a surgeon to have a better view to determine if the bone is healing or fusing properly.

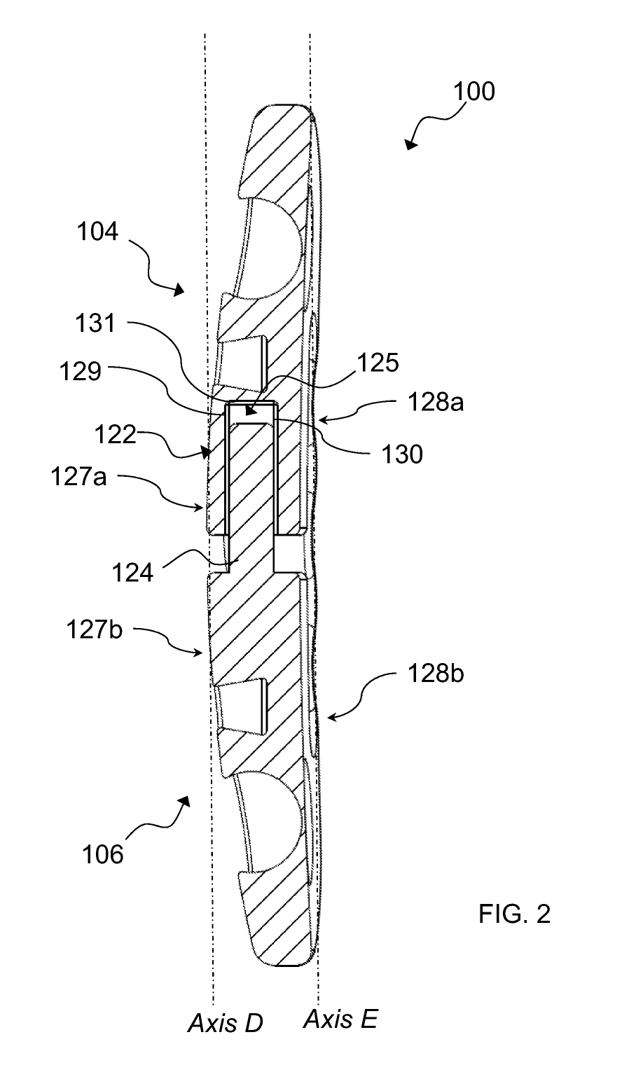

Referring to FIG. 2, a cross sectional side view of the plate 100 is shown. In certain embodiments the first and second plate members 104 and 106 may have a convex anterior surface 127a and 127b that extend along a longitudinal axis D. The first and second plate members 104 and 106 may have a generally concave posterior surface 128a and 128b that extend along a longitudinal axis E. In certain embodiments first plate member 104 may be slidably mated with the second plate member 106. The first plate member 104 may have a receiver segment 122 that defines a longitudinal portal 125. The longitudinal portal 125 may be defined by an anterior (front) wall 129, a posterior (rear) wall 130, a top wall 131 and two side walls 132 and 133 (FIG. 1). The longitudinal portal 125 may be open on a first end and closed on an opposite second end. The longitudinal portal 125 may be dimensioned to at least partially receive a slider segment 124 of the second plate member 106. In certain embodiments the slider segment 124 and the longitudinal portal 125 may be dimensioned to allow for a gap which may allow the first plate member 104 to pivot relative to the second plate member 106. The slider segment 124 may be generally rectangular in shape and may have a proximal and distal end portions. The proximal end portion may have a reduced profile or lead in to aid insertion into the longitudinal portal. The slider segment 124 may slide within the longitudinal portal 125 as the plate 100 is expanded or compressed. Other configurations for the slider segment 124 and the longitudinal portal 125 are possible, as will described in greater detail later.

The plate 100 (or any of the other plate embodiments described herein) may contain instrumentation slots 114a and 114b. In certain embodiments instrumentation slots 114a and 114b may extend partially into the respective first and second plate members 104 and 106. In other embodiments instrumentation slots 114a and 114b may extend through the respective first and second plate members 104 and 106 of plate 100. Instrumentation slots 114a and 114b may be located along center line of plate 100. In certain embodiments the instrumentation slots 114a and 114b may have a race track shape and dimensioned to receive an insertion instrument (not shown) which may be used to hold the plate 100 as it is inserted.

The first and second elastomeric members 108 and 110 members may be placed in tension so the plate 100 compresses one or more boney structures to which the plate 100 is attached. As the distance first and second elastomeric members 108 and 110 are expanded or stretched increases, the resulting compressive force created may also increase. The elastomeric members 108 and 110 may follow the equation “F=−k×”, wherein “F” represents the force on the elastomeric members 108 and 110, “k” is a spring constant of first and second elastomeric members 108 and 110 and “x” is the displacement of first and second elastomeric members 108 and 110. The material, thickness and geometry of the first and second elastomeric members 108 and 110 may be varied to increase their stiffness. In certain embodiments the first and second elastomeric members 108 and 110 may be interchangeable with elastomeric members of varying materials, lengths and geometries.

In certain embodiments the first and second plate members 104 and 106 may be manufactured from the same or dissimilar materials. The first and second plate members 104 and 106 may be manufactured from as nitinol, titanium, stainless steel, elastomers, polymers or other biocompatible materials. The first and second plate members 104 and 106 may be cast, machined, molded or manufactured from any combination of commonly known manufacturing processes.

In certain embodiments the respective coupling members 101a, 101b, 102a and 102b may be dimensioned to receive the elastomeric members 108 and 110. In certain embodiments the elastomeric members 108 and 110 may be secured to the respective coupling members 101a, 101b, 102a and 102b using adhesives or mechanical means, such as knots, crimping or mechanical fasteners. The elastomeric members 108 and 110 may also be secured to the respective coupling members 101a, 101b, 102a and 102b by insert molding or other common assembly methods.

In certain embodiments first and second elastomeric members 108 and 110 may be composed of a solid material or may include a cord that is twisted, weaved or braided. The first and second elastomeric members 108 and 110 may be molded, extruded or spun utilizing common manufacturing equipment and techniques. In certain embodiments the first and second elastomeric members 108 and 110 may include materials such as polyesters, polyolefins, nylons or silicone other polymeric materials. Materials with greater elongation may also be used, such as, polybutadiene, polyisoprene, polychloroprene or other elastomers. A combination of materials may be incorporated into the first and second elastomeric members 108 and 110. In certain embodiments a radio oqapue material, such as a metal, may be weaved or incorporated into the first and second elastomeric members 108 and 110. The radio opaque material may allow a surgeon to view the first and second elastomeric members 108 and 110 on an X-ray or fluoroscopy image.

The first and second elastomeric members 108 and 110 may offer several advantages over conventional coil springs. The first and second elastomeric members 108 and 110 do not extend in a helical fashion around a central axis, but extend in a generally longitudinal direction. A core or coil is created when a spring extends in a helical fashion around a central axis. This core or coil takes up valuable space, especially for a small implant such as an orthopedic plate. Also the larger the force required, the larger the coil and more space that is consumed. The first and second elastomeric members 108 and 110 are designed to exert a maximum force while consuming a minimum amount of space, not only to minimize the size and thickness of the plate, but to allow a surgeon a better view the anatomy to which the plate is attached. Another advantage of first and second elastomeric members 108 and 110 is that their low profile does not interfere with neighboring anatomy which may be impinged. Coils tend to collapse on themselves, thus trapping or impinging neighboring anatomy which may cause pain or damage to a patient. Tissue may also have the tendency to grow within the core or in-between the coils, thus interfering with the function of the plate. The first and second elastomeric members 108 and 110 do not have a core and are designed not to compress against each other which may impinge or trap tissue that is near the plate 100.

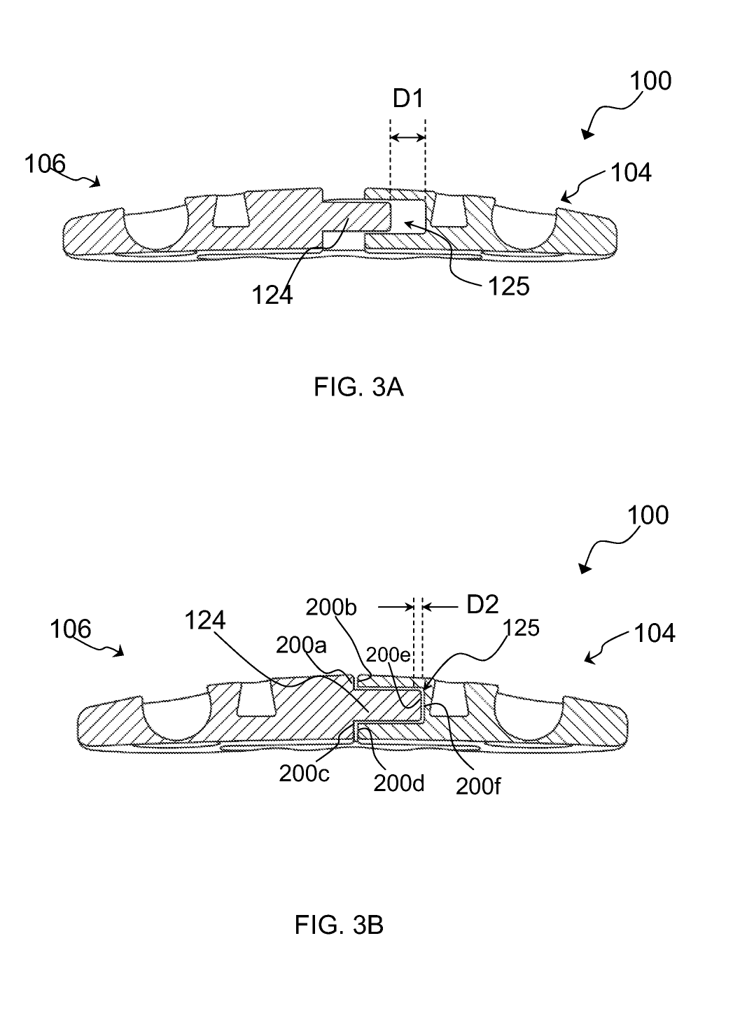

Turning now to FIGS. 3A and 3B a cross sectional side view of the plate 100 is shown illustrating the first plate member 104 and the second plate member 106 in an expanded first position. A force may be applied to the plate 100 to expand the plate 100 a certain distance or to achieve a certain force prior to implanting or attaching the plate 100 to a pair of vertebrae. When a distraction force is placed on the plate, the force may cause the elastomeric members 108 and 110 to elongate. The distance D1 may be used to represent a distance between the first and second plate members 104 and 106 when the plate 100 is expanded. After the plate 100 is inserted and fixed to a pair of vertebrae (or other boney structure) the distraction force may be removed.

A distance D2 may be used to represent a distance between the first and second plate members 104 and 106 when the distraction force is removed from the plate 100. Once the distraction force is removed, the plate 100 may transfer a compressive force to the vertebrae as the first and second elastomeric members 108 and 110 urge the first and second plate members 104 and 106 closer together. The plate 100 may actively compress the vertebrae together, which may result in an improved fusion. The plate 100 may also compress a graft or an implant (not shown) that is located between the two vertebrae, which may help prevent expulsion of the graft or implant. The elastomeric members 108 and 110 may be capable of exerting a compression force on the vertebrae (or graft/implant) which is greater than a force normally acting on the vertebrae when a patient is in a standing position. In certain embodiments the elastomeric members 108 and 110 may exert a compressive force of less than 10 lbs to more than 50 lbs. In certain embodiments the distance D2 may be less than D1, but may still be greater than a distance between the first and second plate members 104 and 106 when the plate 100 is in a pre-expanded or neutral position.

After the plate 100 is attached to the adjacent vertebrae, settling may occur, as the vertebrae move closer together. This may cause the first and second plate members to move closer together. The plate 100 may have one or more abutment surfaces 200a-200f which may prevent the first and second the first plate members 104 and 106 from moving closer together. The abutment surfaces may prevent the vertebrae from collapsing together and may help maintain disc height (the distance between adjacent vertebrae). In some embodiments an end wall 200e of the slider segment 124 may contact a back wall 200f of the longitudinal portal 125 to prevent the first and second plate members 104 and 106 from moving closer together. In other embodiments a bottom surface 200b and 200d of the first plate member 104 may contact a top surface 200a and 200c of the second plate member 106 to prevent further compression of the plate.

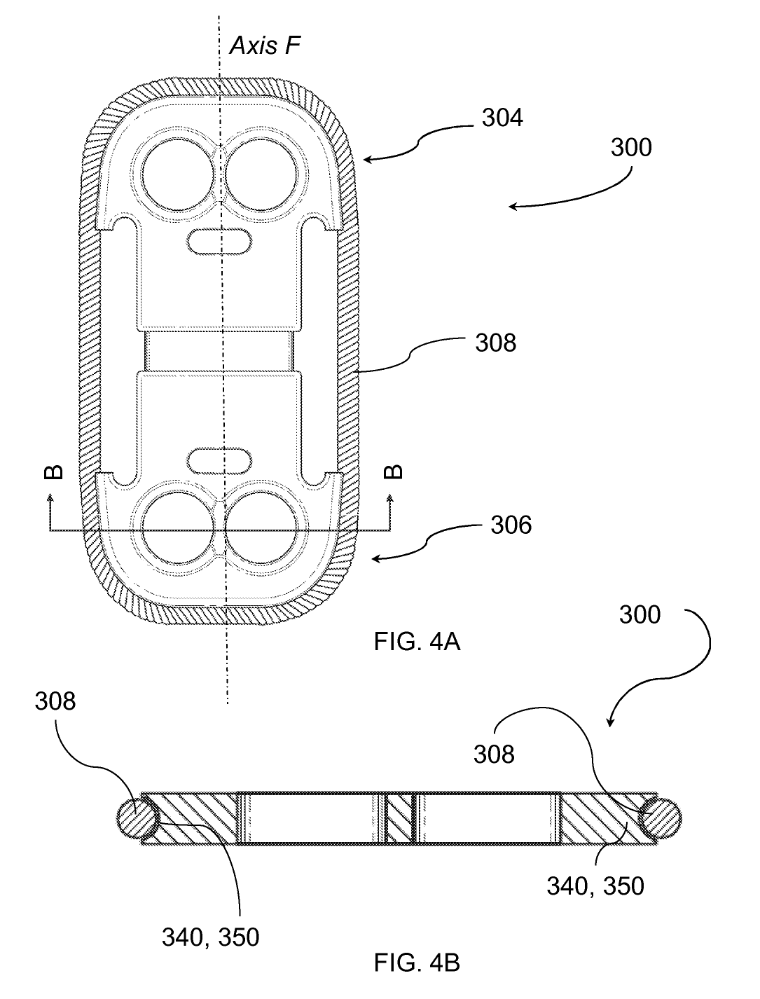

Turning now to FIGS. 4A and 4B, a front view of an alternative embodiment of a plate 300 is shown. The plate 300 may extend generally along a longitudinal axis F and may include a first plate member 304, a second plate member 306 and one or more elastomeric members 308. The first and second plate members 304 and 306 may be slidably coupled as previously described for the first and second plate members 104 and 106. The plate 300 may be fastened to one or more adjacent (or non adjacent) boney structures, such as vertebrae, and the elastomeric member 308 may force the vertebrae closer together. The elastomeric member 308 may aid in compressing a graft or an implant located between the adjacent vertebrae to promote fusion. The plate 300 may also be used to stabilize boney fractures which may or may not have an implant or graft in-between to promote fusion or improve healing. For example, the elastomeric member 308 may force long bone structures on opposing sides of a fracture together to promote fusion. Accordingly, the first plate member 304, the second plate member 306 and the elastomeric member 308 may interact to provide the proper stabilization forces required to support or fuse boney structures.

The plate 300 and the elastomeric member 308 may have similar features, functions and advantages as the plate 100 and the elastomeric members 106 and 108, but in certain embodiments the elastomeric member 308 may include a continuous loop that extends around the perimeter of the first and second plate members 304 and 306. The outer surface of the first and second plate members 304 and 306 may have a groove or retaining feature 340 and 350 respectively, that extends around the perimeter of the plate 300, as shown in FIG. 4B. The grooves 340 and 350 may aid in retaining the elastomeric member 308 coupled to the plate 300. In other embodiments the elastomeric member 308 may be coupled or bonded to the plate by mechanical or chemical methods.

The elastomeric member 308 may be composed of various metallic wires, elastomers and polymers or combinations thereof depending on the desired compressive or extension force required. Similar materials and methods of manufacture may be used for elastomeric member 308 as elastomeric members 108 and 110. In certain embodiments the elastomeric member 308 may be a continuous twist, braid or weave (as shown) or may be a cord with its ends tied or welded together to create a loop. In other embodiments the elastomeric member 308 may be composed of a molded or extruded solid polymer.

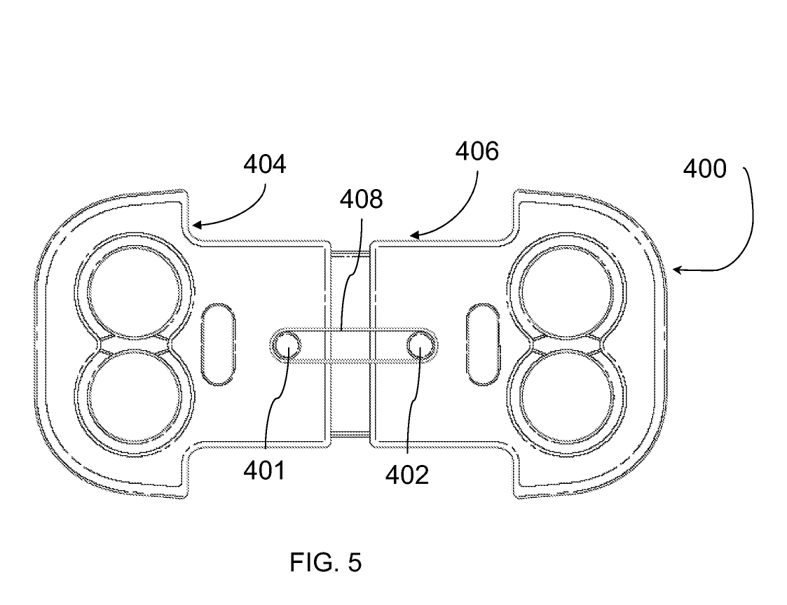

The elastomeric member 308 may also be used in other embodiments, for example, FIG. 5 illustrates an alternative embodiment of a plate 400 with an elastomeric member 408 wrapping around one or more retaining features or protrusions 401 and 402. The protrusions 401 and 402 may be positioned on or within a top surface of a first and second plate members 404 and 406, respectively. In certain embodiments the protrusions 401 and 402 may include posts with notches or grooves (not shown) to capture the elastomeric member 408. Alternatively, the protrusions 401 and 402 may have configurations such as hooks or tabs which aid in securing the elastomeric member 408 to plate 400. The elastomeric member 408, does not necessarily have to be a loop, but may be single member as previously described for elastomeric members 108 and 110.

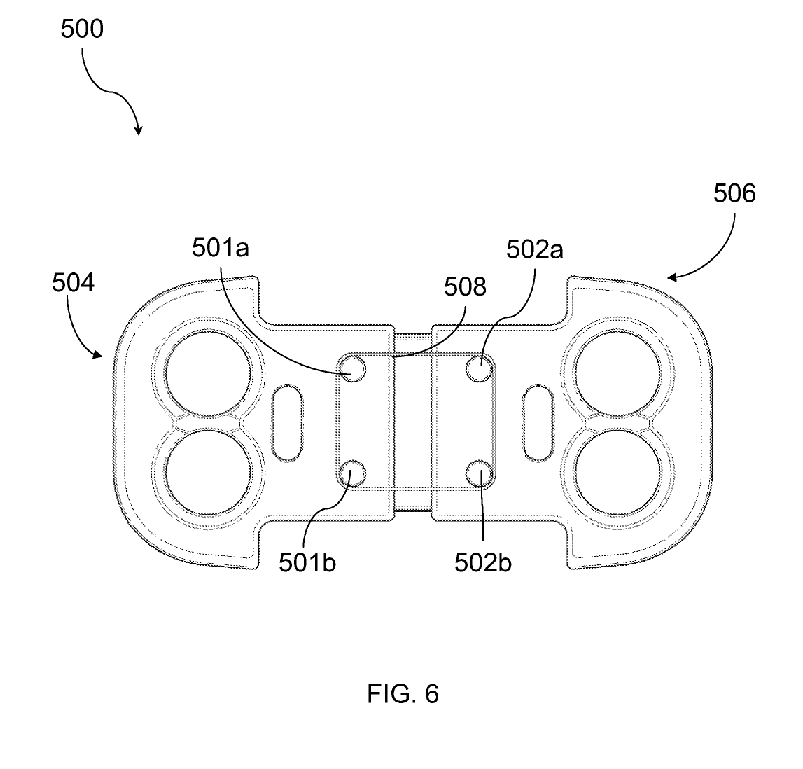

Referring to FIG. 6, yet another embodiment of a plate 500 is shown illustrating another possible embodiment of an elastomeric member 508. The plate 500 may be similar to plate 400, however plate 500 may include several retaining features or protrusions 501a, 501b, 502a and 502b which may better distribute forces of the elastomeric member 508 to the plate 500. FIG. 6 illustrates a front view of the plate 500 with the elastomeric member 508 coupled to protrusions 501a and 501b on a first plate member 504 and coupled to two protrusions 502a and 502b on a second plate member 506. Although the protrusions 501 and 402 in the above described embodiments are located toward the distal end of their respective first and second plate members 406 (and 506) and 408 (and 508) the protrusions 401 and 402 (and 501a, 501b, 502a and 502b) may be located anywhere on the top surface of plate 400 or 500. In certain embodiments the elastomeric members 308, 408 or 508 may be positioned within a groove or a housing as not to interfere with a patient's anatomy (not shown).

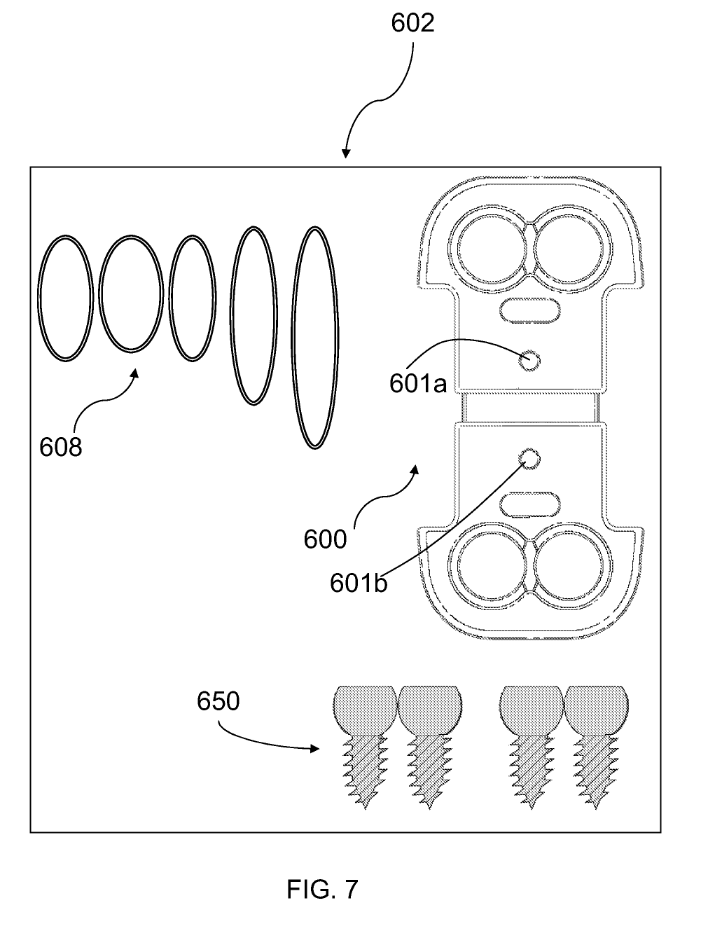

Referring now to FIG. 7, a surgical instrument kit 602 is shown which may incorporate any of the embodiments disclosed herein. In certain embodiments the surgical instrument kit 602 may include a plate 600, a plurality of elastomeric members 608, and a plurality of bone anchors 650. The plate 600 may include any of the plates 100, 300, 400 or 500 as previously mentioned. The plurality of elastomeric members 608 may each have different lengths, thickness or stiffnesses. A surgeon may gradually increase a force exerted by the plate 600 to a bony structure by adding additional elastomeric members 608 to the plate 600 either during the procedure or post operatively as needed. The elastomeric members 608 may be coupled to one or more attachment features 601a and 601b that may be located on a top surface of the plate 600. The plurality of bone anchors 650 may include bone screws to secure the plate 600 to a boney structure. The bone anchors 650, are not limited to screws, but may include other bone anchors such as hooks, pins, plates, staples, or other fastners that are commonly known and used in the orthopedic industry. The kit 602 may give a surgeon the freedom to determine or vary the amount of force the plate 600 should exert on a boney structure depending on the specific patient anatomy or other conditions of the surgery.

Other embodiments for a surgical bone plate may include:

1. A surgical bone plate comprising:

a first plate member extending along a longitudinal axis, having a first and second laterally offset coupling members, a plurality of bone screw holes an a receiving segment with a longitudinal portal;

a second plate member positioned along the longitudinal axis, having a third and fourth laterally offset coupling members, a plurality of bone screw holes and a slider segment dimensioned to fit within the longitudinal portal of the first plate member;

a first elastomeric member extending between and coupled to the first and third laterally offset coupling members; and

a second elastomeric member extending between and coupled to the third and fourth laterally offset coupling members, wherein the first and second elastomeric members are generally parallel to the longitudinal axis and laterally spaced apart from the receiving segment and the slider segment to define a first and second windows.

2. The surgical bone plate of claim 1 wherein the first and second plate members each have a top surface that is curved along the longitudinal axis.

3. The surgical bone plate of claim 1 wherein the first and second plate members each have a bottom bone contacting surface that is curved along the longitudinal axis.

4. The surgical bone plate of claim 1 wherein the first and second plate members each have at least one instrumentation recess.

5. The surgical bone plate of claim 1 wherein the first and second elastomeric members are braided.

6. The surgical bone plate of claim 1 wherein the plurality of bone screw holes of the first or the second plate members overlap.

7. The surgical bone plate of claim 1 wherein the first and second plate members are composed of a radio lucent material.

8. The surgical bone plate of claim 7 further comprising at least one radio opaque marker positioned within the first and second plate members.

9. The surgical bone plate system of claim 1 wherein the first and second plate members include a one way ratcheting mechanism.

10. The surgical bone plate of claim 1 wherein the first and second elastomeric members are composed at least partially of a polyester material.

11. The surgical bone plate of claim 1 wherein the first and second elastomeric members are composed at least partially of a polyolefin material.

12. The surgical bone plate of claim 1 wherein the first and second elastomeric members are composed at least partially of a nylon material.

13. The surgical bone plate of claim 1 wherein the first and second elastomeric members are composed of a material selected from the group consisting of polybutadiene, polyisoprene and polychloroprene.

Still other embodiments for a surgical bone plate may include:

1. A surgical bone plate comprising:

a first plate member extending along a longitudinal axis, having a top surface, a bottom surface an outer side surface and a receiving segment with a longitudinal portal;

a second plate member positioned along the longitudinal axis, having a top surface, a bottom surface, an outer side surface and a slider segment slidably positioned within the longitudinal portal of the first plate member;

a groove extending along the outer side surface of the first and second plate members

an elastomeric member positioned at least partially within the groove wherein the elastomeric member has two legs that are generally parallel to the longitudinal axis and are laterally spaced apart from the receiving segment and the slider segment to define a first and second windows.

2. The surgical bone plate of claim 1 wherein the top surfaces of the first and second plate members are curved along the longitudinal axis.

3. The surgical surgical bone plate of claim 1 wherein the top surfaces of the first and second plate members are curved along the longitudinal axis.

4. The surgical bone plate of claim 1 wherein the first and second plate members each have at least one instrumentation recess.

5. The surgical bone plate of claim 1 wherein the first and second elastomeric members are braided.

6. The surgical bone plate of claim 5 wherein the first and second elastomeric members are composed of a plurality of radio lucent and radio opaque fibers.

7. The surgical bone plate system of claim 1 wherein the first and second plate members include a one way ratcheting mechanism.

8. The bone plate of claim 1 wherein the first and second elastomeric members are composed at least partially of a polyester material.

11. The surgical bone plate of claim 1 wherein the first and second elastomeric members are composed at least partially of a polyolefin material.

12. The surgical bone plate of claim 1 wherein the first and second elastomeric members are composed at least partially of a nylon material.

13. The surgical bone plate of claim 1 wherein the first and second elastomeric members are composed of a material selected from the group consisting of polybutadiene, polyisoprene and polychloroprene.

14. The surgical bone plate of claim 1 wherein the first and second elastomeric members are composed at least partially of silicone.

Still other embodiments for a surgical bone plate may include:

1. A bone plate comprising:

a first plate member extending along a longitudinal axis, having a top surface, a bottom surface and a receiving segment with a longitudinal portal;

a second plate member positioned along the longitudinal axis, having a top surface, a bottom surface and a slider segment slidably positioned within the longitudinal portal of the first plate member;

a first protrusion located on the top surface of the first plate member and a second protrusion located on the top surface of the second plate member; and

a first elastomeric member coupled to the first and second protrusions.

2. The bone plate of claim 1 further comprising a third protrusion located on the top surface of the first plate member, a fourth protrusion located on the top surface of the second plate member and a second elastomeric member coupled to the third and fourth protrusions.

3. The bone plate of claim 1 wherein the top surfaces of the first and second plate members are curved along the longitudinal axis.

4. The bone plate of claim 1 wherein the top surfaces of the first and second plate members are curved along the longitudinal axis.

5. The bone plate of claim 1 wherein the first and second plate members each have at least one instrumentation recess.

6. The bone plate of claim 1 wherein the first and second elastomeric members are braided.

7. The bone plate of claim 5 wherein the first and second elastomeric members are composed of a plurality of radio lucent and radio opaque fibers.

8. The bone plate system of claim 1 wherein the first and second plate members include a one way ratcheting mechanism.

9. The bone plate of claim 1 wherein the first and second elastomeric members are composed at least partially of a polyester material.

10. The bone plate of claim 1 wherein the first and second elastomeric members are composed at least partially of a polyolefin material.

Other embodiments for a surgical bone plate kit may include:

1. A kit for stabilizing one or more bone structures, comprising:

a first plate member extending along a longitudinal axis, having a top surface, a bottom surface and a receiving segment with a longitudinal portal;

a second plate member positioned along the longitudinal axis, having a top surface, a bottom surface and a slider segment slidably positioned within the longitudinal portal of the first plate member;

a retaining feature located on the top surface of the first plate member and a retaining feature located on the top surface of the second plate member; and

a plurality of elastomeric members of varying stiffness.

2. The kit of claim 1 further comprising a plurality of elastomeric members of varying length.

3. The bone plate of claim 1 wherein the top surfaces of the first and second plate members are curved along the longitudinal axis.

4. The bone plate of claim 1 wherein the top surfaces of the first and second plate members are curved along the longitudinal axis.

5. The bone plate of claim 1 wherein the first and second plate members each have at least one instrumentation recess.

6. The bone plate of claim 1 wherein the first and second elastomeric members are braided.

7. The bone plate of claim 5 wherein the first and second elastomeric members are composed of a plurality of radio lucent and radio opaque fibers.

8. The bone plate system of claim 1 wherein the first and second plate members include a one way ratcheting mechanism.

9. The bone plate of claim 1 wherein the first and second elastomeric members are composed at least partially of a polyester material.

10. The bone plate of claim 1 wherein the first and second elastomeric members are composed at least partially of a polyolefin material.

11. The bone plate of claim 1 wherein the first and second elastomeric members are composed at least partially of a nylon material.

12. The bone plate of claim 1 wherein the first and second elastomeric members are composed of a material selected from the group consisting of polybutadiene, polyisoprene and polychloroprene.

13. The bone plate of claim 1 wherein the first and second elastomeric members are composed at least partially of silicone.

The foregoing details provided regarding the embodiments of the invention have been presented primarily for the purposes of illustration and description. The details and drawings are not intended to be exhaustive listing of potential embodiments, nor should they limit the invention to the precise forms disclosed. Many modifications, combinations, and variations of the various disclosed embodiments are possible in light of the above teachings while still remaining within the subject matter of the invention. It is intended that the scope of the invention is only limited by the Claims appended hereto. The abstract is in no way intended to limit the scope of the invention.

What is claimed is:

1. A surgical bone plate system comprising:
a first plate member extending along a longitudinal axis, having a first and second laterally offset coupling members and a receiving segment with a longitudinal portal, the first plate member defining a plurality of bores extending through a top and bottom surface of the first plate member;
a second plate member positioned along the longitudinal axis, having a third and fourth laterally offset coupling members, and a slider segment dimensioned to fit within the longitudinal portal of the first plate member, the second plate member defining a plurality of bores extending through a top and bottom surface of the second plate member;
a first elastomeric member extending between and coupled to the first and third laterally offset coupling members;
a second elastomeric member extending between and coupled to the second and fourth laterally offset coupling members, wherein the first and second elastomeric members are generally parallel to the longitudinal axis and laterally spaced apart from the receiving segment and the slider segment to define a first and second windows; and
a plurality of bone anchors at least partially received within the plurality of bores of the first and second plate members.

2. The surgical bone plate system of claim 1 wherein the top surface of the first and second plate members is curved along the longitudinal axis.

3. The surgical bone plate system of claim 1 wherein the bottom surface of the first and second plate members is curved along the longitudinal axis.

4. The surgical bone plate system of claim 1 wherein the first and second plate members each have at least one instrumentation recess.

5. The surgical bone plate of claim 1 wherein the first and second elastomeric members are braided.

6. The surgical bone plate of claim 5 wherein the first and second elastomeric members are composed at least partially of a polyester material.

7. The surgical bone plate of claim 1 wherein the first and second elastomeric members are composed at least partially of a polyolefin material.

8. The surgical bone plate of claim 1 wherein the first and second elastomeric members are composed at least partially of a nylon material.

9. The surgical bone plate of claim 1 wherein the first and second elastomeric members are composed of a material selected from the group consisting of polybutadiene, polyisoprene and polychloroprene.

10. A surgical bone plate comprising:
a first plate member extending along a longitudinal axis, having a top surface, a bottom bone contacting surface an outer side surface and a receiving segment with a longitudinal portal;
a second plate member positioned along the longitudinal axis, having a top surface, a bottom bone contacting surface, an outer side surface and a slider segment slidably positioned within the longitudinal portal of the first plate member;
a groove extending along the outer side surface of the first and second plate members
an elastomeric member positioned at least partially within the groove wherein the elastomeric member has two legs that are generally parallel to the longitudinal axis and are laterally spaced apart from the receiving segment and the slider segment to define a first and second windows.

11. The surgical bone plate of claim 1 wherein the first and second plate members each have at least one instrumentation recess.

12. The surgical bone plate of claim 1 wherein the elastomeric member is braided.

13. The surgical bone plate of claim 10 wherein the elastomeric member is composed of a plurality of radio lucent and radio opaque fibers.

14. The surgical bone plate of claim 10 wherein the first and second elastomeric members are composed at least partially of a polyester material.

15. A surgical bone plate comprising:
a first plate member extending along a longitudinal axis, having a top surface, a bottom surface and a receiving segment with a longitudinal portal;
a second plate member positioned along the longitudinal axis, having a top surface, a bottom surface and a slider segment slidably positioned within the longitudinal portal of the first plate member;
a first protrusion located on the top surface of the first plate member and a second protrusion located on the top surface of the second plate member; and
a first elastomeric member coupled to the first and second protrusions.

16. A kit for stabilizing one or more bone structures, comprising:
a first plate member extending along a longitudinal axis, having a top surface, a bottom surface and a receiving segment with a longitudinal portal;
a second plate member positioned along the longitudinal axis, having a top surface, a bottom surface and a slider segment slidably positioned within the longitudinal portal of the first plate member;
a retaining feature located on the top surface of the first plate member and a retaining feature located on the top surface of the second plate member;
a plurality of bone anchors; and
a plurality of elastomeric members of varying stiffness.

17. A method of compressing adjacent boney structures, comprising the steps of:
providing a plate having a first and second plate members coupled together with at least one elastomeric member;
elongating the elastomeric member by applying a distraction force to the plate;
sliding the first plate member relative to the second plate member;
fastening the first plate member to a first boney structure with a first and second anchors;
fastening the second plate member to a second boney structure with a third and fourth anchors; and
removing the distraction force from the plate.

18. A method of compressing adjacent boney structures, comprising the steps of:
providing a plate having a first and second plate members coupled together with at least one elastomeric member;
coupling an instrument to the first and second plate members;
elongating the elastomeric member by applying a distraction force to the plate with the instrument;
sliding the first plate member relative to the second plate member;
fastening the first plate member to a first boney structure with a first and second anchors;
fastening the second plate member to a second boney structure with a third and fourth anchors; and
removing the instrument from the plate.

19. A method of compressing adjacent boney structures, comprising the steps of:
providing a plate having a first plate member with at least one retaining feature and a second plate member with at least one retaining feature;
sliding the first plate member axially relative to the second plate member;
fastening the first plate member to a first boney structure with a first and second anchors;
fastening the second plate member to a second boney structure with a third and fourth anchors;
coupling a first elastomeric member having a first stiffness to the retaining features of the first and second plate members.

20. The method of claim 19 further comprising the steps of:
coupling a second elastomeric member having a second stiffness to the retaining features of the first and second plate members.

21. A method of compressing adjacent boney structures, comprising the steps of:
providing a plate having a first plate member with at least one retaining feature and a second plate member with at least one retaining feature;
sliding the first plate member axially relative to the second plate member;
fastening the first plate member to a first boney structure with a first and second anchors;
fastening the second plate member to a second boney structure with a third and fourth anchors;
expanding an elastomeric having a first stiffness around the retaining features of the first and second plate members.

22. The method of claim 21 further comprising the steps of:
coupling a second elastomeric member having a second stiffness to the retaining features of the first and second plate members.

23. A method of compressing adjacent boney structures, comprising the steps of:
providing a plate having a first and second plate members;
sliding the first plate member axially relative to the second plate member;
fastening the first plate member to a first boney structure with a first and second anchors;
fastening the second plate member to a second boney structure with a third and fourth anchors; and
expanding an elastomeric member around the perimeter of the plate.
